(19) United States
(12) Patent Application Publication
(10) Pub. No.: US 2006/0142621 A1
(43) Pub. Date: Jun. 29, 2006

(54) Bismuth dithiocarbamate compounds and uses thereof

(76) Inventor: Edward R T Tiekink, SINGAPORE (SG)

Correspondence Address: OSTROLENK FABER GERB & SOFFEN, 1180 AVENUE OF THE AMERICAS, NEW YORK, NY, 100368403, US

(21) Appl. No.: 10/543,899

(22) PCT Filed: Jan. 2, 2004
(86) PCT No.: PCT/US04/00028

§ 371 Date: Oct. 27, 2005

Related U.S. Application Data
(60) Provisional application No. 60/443,165, filed on Jan. 29, 2003.

(51) Int. Cl. C07C 17/25

(52) U.S. Cl. 570/157

(57) ABSTRACT

The present invention relates to bismuth dithiocarbamate compounds of general formula I: wherein R and R1 are the same or different, and each is an alkyl, substituted alkyl, aryl or a substituted aryl; X is a halide or a pseudo-halide; and n is either 2 or 3, for use as an active pharmaceutical substance. In particular, they can be used as anti-tumour agents and for the treatment of various forms of tumours. Also provided are some novel bismuth dithiocarbamate compounds and the process for preparing these compounds.

### FIELD OF THE INVENTION

The present invention relates to bismuth dithiocarbamate compounds. In particular, to bismuth dithiocarbamate compounds for use in therapy.

### BACKGROUND OF THE INVENTION

Chemotherapy is at the forefront of tackling cancer. Chemotherapeutic agents currently employed suffer from a lack of efficacy and are known to cause deleterious side-effects in patients. Amongst the important drugs used to treat cancer, some of the most effective are metal-based.

Metal-containing compounds may offer certain advantages over purely organic compounds in drug therapy. For example, co-ordination of an organic molecule to a metal centre may alter the normal metabolic pathway and/or may lead to a slow release mechanism for delivery of the organic molecule i.e. the metal complex may function as a pro-drug.

In this regard, the widely used drug, cisplatin, [(NH₃)₂PtCl₂], is particularly notable. While cisplatin is effective against certain cancers such as head, neck and testicular cancers, it lacks selectivity for tumour tissue, which leads to severe side effects. Notwithstanding the widespread applications of platinum anticancer drugs, there is still a large need for the development of novel metal-based compounds with unprecedented features. Some known metal-based compounds have been proven to have effect only on limited types of cancer while other known metal-based compounds have not been used in therapy.

In the medical field, bismuth compounds are generally only used to relieve diaper (nappy) rash, treat burns as well as to treat gastric disorders such as diarrhoea and even ulcers. At present, bismuth subcitrate and bismuth subsalycilate are used to treat such medical conditions, of which both are Bi (III) ions. Their empirical formulae are often given as K₃(NH)₄[Bi₆O₃(OH)₅(H_{clt})₄] (Merck Index, 1989, 11^{th} edition, p 197) and OC₆H₄COOBiO (Sun, Li et. al., 1997, Chem. Ber./Recuel. Vol 130, p 675) respectively.

A series of bismuth xanthates with general formula Bi(S₂COR)₃ have also been synthesised recently (M. J. Cox and E. R. T. Tiekink, 1998, Z. Kristallogr., 213:487-492). However, the biological activity of these compounds has not been conducted yet. In particular, bismuth tris(alkylxanthate), Bi(S₂COR)₃ where R is methyl, ethyl or isopropyl.

Other bismuth compounds such as bismuth tris(methyl-n-hexyl-dithiocarbamate), Bi(S₂CN(Me)Hex)₃ and bismuth (III) tris(n,n-diethyldithiocarbamate), Bi(S₂CNEt₂)₃ have only been described for crystallographic studies (Koh et. al., 2003, Chem. Mater., 15, 4544-4554; Monteira et. al., 2001, Chem. Mater., 13, 2103-2111) and their use as precursors for Bi₂S₃ nanoparticles.

Thus there is a need in the art for alternative compounds to be used in therapy, especially as anti-cancer agents. Further, the potential of bismuth as anti-cancer drugs has not been fully explored.

### SUMMARY OF THE INVENTION

The present invention addresses the problems above, and in particular provides new use for bismuth dithiocarbamate compounds.

According to one aspect, the invention provides a compound of general formula I: 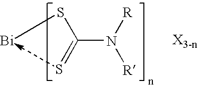
wherein R and R¹ are the same or different, and each is an alkyl, substituted alkyl, aryl or a substituted aryl; X is a halide or a pseudo-halide; and n is either 2 or 3; for use as an active pharmaceutical substance.

Further, the invention also provides a compound of formula I, wherein n is 3 and R and R¹ are both ethyl, for use as an active pharmaceutical substance.

According to another aspect of the present invention, the compound of general formula I can be used in the manufacture of a medicament for therapeutic application as an anti-tumour agent. In particular, the compound having general formula I, wherein n is 3, R and R¹ are both ethyl.

The compounds may be used for the treatment of tumor including but not limited to breast cancer, ovarian cancer, melanoma, renal cancer and non-small cell lung cancer. The tumor can be animal or human tumor.

According to a further aspect, a pharmaceutical composition comprising the bismuth dithiocarbamate compounds of general formula I is provided. In particular, a pharmaceutical composition comprising the compound Bi(S₂CNEt₂)₃. The pharmaceutical compositions optionally comprise a pharmaceutical acceptable diluent and/or carrier.

Another aspect of the invention is a method for treating tumor comprising the administration of compound of general formula I: 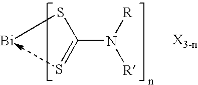
wherein R and R¹ are the same or different, and each is an alkyl, substituted alkyl, aryl or a substituted aryl; X is a halide or a pseudo-halide; and n is either 2 or 3. In particular, n is 3 and R and R¹ are both ethyl.

Further, the above-mentioned method wherein the tumor is animal or human tumor and includes but is not limited to breast cancer, ovarian cancer, melanoma, renal cancer and non-small cell lung cancer.

According to another aspect, the invention provides novel compounds of the general formula I: 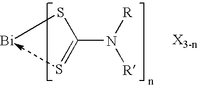
wherein R and R¹ are the same or different, and each is an alkyl, substituted alkyl, aryl or a substituted aryl; X is a halide or a pseudo-halide; and n is either 2 or 3, with the proviso that when n is 3, R and R¹ are not ethyl (Et), and that when n is 3, R is not methyl (Me) and R¹ is not hexyl (Hex) or vice versa.

In particular, the invention provides new compounds included in the general formula I, wherein n is 2. These compounds are indicated with general formula II: 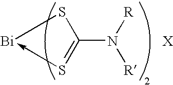
wherein R and R′ are both (CH₂)₂ and X is Cl.

Further, the compounds of formula II may be used in therapy as well as in other fields such as the preparation of nanoparticles.

Another aspect of the present invention is the process for preparing the compound of formula II, which comprises mixing a bismuth salt, alcohol and carbodithioic acid to obtain a mixture and then dissolving the precipitate obtained from the mixture in a halogen-substituted alkane and organic nitrile to further obtain a solution, followed by drying the said solution to recover an amorphous form of compound of formula II.

In particular, the bismuth salt is anhydrous bismuth (III) chloride, the alcohol is ethanol, the acid is 1-pyrrolidinecarbodithioic acid, NH₄S₂CNC₄H₈, the halogen-substituted alkane is chloroform, CHCl₃, and the organic nitrile is acetonitrile, C₂H₃N. Further, the halogen-substituted alkane and organic nitrile are mixed in a 1:1 volume ratio.

### BRIEF DESCRIPTION OF THE FIGURES

FIG. 1a: The cytotoxic effects of Bi(SCNEt₂)₃ (referred to as Bi(dedtc)₃ in figure) on the ovarian cancer cell line (OVCAR).

FIG. 1b: The cytotoxic effects of Bi(S₂CN(CH₂)₄)₂Cl (referred to as Bi(pydtc)₂Cl in figure) on the ovarian cancer cell line (OVCAR).

FIG. 2a: The cytotoxic effects of Bi(SCNEt₂)₃ (referred to as Bi(dedtc)₃ in figure) on A-498 cell line.

FIG. 2b: The cytotoxic effects of Bi(S₂CN(CH₂)₄)₂Cl (referred to as Bi(pydtc)₂Cl in figure) on A-498 cell line.

FIG. 3a: The cytotoxic effects of Bi(SCNEt₂)₃ (referred to as Bi(dedtc)₃ in figure) on HF cell line.

FIG. 3b: The cytotoxic effects of Bi(S₂CN(CH₂)₄)₂Cl (referred to as Bi(pydtc)₂Cl In figure) on HF cell line.

FIG. 4a: The cytotoxic effects of Bi(SCNEt₂)₃ (referred to as Bi(dedtc)₃ in figure) on NCl-H1299 cell line.

FIG. 4b: The cytotoxic effects of Bi(S₂CN(CH₂)₄)₂Cl (referred to as Bi(pydtc)₂Cl in figure) on NCl-H1299 cell line.

FIG. 5a: The cytotoxic effects of Bi(SCNEt₂)₃ (referred to as Bi(dedtc)₃ in figure) on HT-29 cell line.

FIG. 5b: The cytotoxic effects of Bi(S₂CN(CH₂)₄)₂Cl (referred to as Bi(pydtc)₂Cl in figure) on HT-29 cell line.

FIG. 6a: The cytotoxic effects of Bi(SCNEt₂)₃ (referred to as Bi(dedtc)₃ in figure) on MRC-5 cell line.

FIG. 6b: The cytotoxic effects of Bi(S₂CN(CH₂)₄)₂Cl (referred to as Bi(pydtc)₂Cl in figure) on MRC-5 cell line.

FIG. 7a: The cytotoxic effects of Bi(SCNEt₂)₃ (referred to as Bi(dedtc)₃ in figure) on MCF-7 cell line.

FIG. 7b: The cytotoxic effects of Bi(S₂CN(CH₂)₄)₂Cl (referred to as Bi(pydtc)₂Cl in figure) on MCF-7 cell line.

FIG. 8: Plot of relative tumour volume (mm³) versus days after administration of Bi(S₂CNEt₂)₃ (test group) and DMSO only (control group) for nude Balb/C mice inoculated with OVCAR (human ovarian cancer).

### DETAILED DESCRIPTION OF THE INVENTION

One aspect of the present invention relates to the use of bismuth dithiocarbamate compounds for use as an active pharmaceutical substance. The general formula of these compounds is as follows: 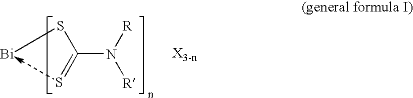
wherein R and R¹ are the same or different, and each is an alkyl, substituted alkyl, aryl or a substituted aryl; X is a halide or a pseudo-halide; and n is either 2 or3.

As used herein, the term ‘alkyl’ refers to a straight or branched, monovalent, saturated aliphatic chain of 1-20 carbon atoms, including normal, iso, neo and tertiary. ‘Alkyl’ includes but is not limited to, methyl, ethyl, propyl, isopropyl, butyl, iso-butyl, sec butyl, tert butyl, amyl, isoamyl, neoamyl, hexyl, isohexyl, neohexyl, heptyl, isoheptyl, neoheptyl, octyl, isooctyl, neooctyl, and the like; cycloalkyl group such as cyclopropyl, cyclobutyl, cyclopentyl, cyclohexyl and the like, the cycloalkyl group may be substituted.

The term ‘aryl’ refers to organic compounds including but not limited to phenyl, biphenyl, naphthyl, furanyl, pyrrolyl, thiophenyl, pyridinyl, indolyl, benzofuranyl, benzothiophenyl, quinolinyl, isoquinolinyl, imidazolyl, thiazolyl, pyrazinyl, primidinyl, purinyl and pteridinyl. The aryl group may be substituted and may include the following compounds:

The term ‘halide’ refers to a family of non-metallic, generally electronegative, elements of group VII of the periodic table. They are all multivalent and have oxidation numbers of −1 (the most common), 1, 3, 5, and 7. Examples of halides are fluorine (F), chlorine (Cl), bromine (Br) and iodine (I). The term pseudo-halide refers to SCN, CN, NCO and the like.

In particular, the compound of general formula I, wherein n is 3 and R and R¹ are both ethyl, for use as an active pharmaceutical substance. This compound is known as bismuth (III) tris(N,N-diethyldithiocarbamate), Bi(S₂CNEt₂)₃. Although this is a known compound (Howard et. al., 1975, Acta Crystallographica., 31,S141 and Raston et. al., 1976, J. Chem. Soc., Dalton Trans., 791), its previous uses are non-medical uses. It has most commonly been used in crystallographic studies. The properties of this compound are as follows based on the preparation of the compound as described in Koh et. al., 2003, Chem. Mater., 15, 4544-4554: ¹H NMR: δ 1.33 [6H, t, J 6.8 Hz, CH₃] and 3.8 [4H, q, J7.2 Hz, CH₂]. {¹H} ¹³C NMR: δ 200.1 (S₂C); 48.3 (CH₂), 12.2 (CH₃). Elemental analysis (%) found: C, 27.57; H, 4.65; N, 6.24; S, 29.99. Calcd: C, 27.53; H, 4.59; N, 6.43; S, 29.44. IR (cm⁻¹): 1481 [ν(C—N)], 984 [ν(C—S)]MS: m/z=505.0 ([Bi(S₂CNEt₂)₂]⁺). Yield 57%;. mp 200-201° C.

A further embodiment of the invention is the use of the compound of general formula I in the manufacture of a medicament for therapeutic application as an anti-tumour agent. These compounds are found to be highly effective with minimal deleterious side effects owing to the high mammalian tolerance of bismuth. The known compound Bi(S₂CNEt₂)₃) is also used in the manufacture of a medicament for therapeutic application as an anti-tumour agent.

These compounds can be used in the treatment of tumours, which can be animal or human tumours. In particular, the compounds can also be used to treat tumours including but not limited to breast cancer, ovarian cancer, melanoma, renal cancer and non-small cell lung cancer.

The compound of general formula I can also be used as a pharmaceutical composition for use in therapy. For example, it is useful for the prevention or reduction of tumours. The invention encompasses the preparation and use of pharmaceutical compositions comprising the compound of general formula I as an active ingredient. Such a pharmaceutical composition may consist of the active ingredient alone, in a form suitable for administration to a subject, or alternatively the pharmaceutical composition may comprise the active ingredient and one or more pharmaceutically acceptable carrier, excipient and/or diluent.

The formulations of the pharmaceutical compositions described herein may be prepared by any method known or hereafter developed in the art. In general, such preparatory methods include the step of bringing the active ingredient into association with a carrier, excipient and/or diluent, and then, if necessary or desirable, shaping or packaging the product into a desired single- or multi-dose unit.

Although the descriptions of pharmaceutical compositions provided herein are principally directed to pharmaceutical compositions that are suitable for administration to humans, the person skilled in the art will understand that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modifications. Subjects to which administration of the pharmaceutical compositions of the invention is contemplated include, but are not limited to, humans and other primates.

Another embodiment of the invention is the method for treating tumours comprising the administration of compound of general formula I: 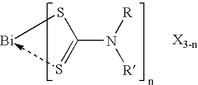
wherein R and R¹ are the same or different, and each is an alkyl, substituted alkyl, aryl or a substituted aryl; X is a halide or a pseudo-halide; and n is either 2 or 3. In particular, the method for treating tumour comprises the administration of the compound of general formula I, with n=3, R and R¹ are both ethyl.

Tumour, as stated in the above-mentioned method includes breast cancer, ovarian cancer, melanoma, renal cancer and non-small cell lung cancer. Further, the tumour is animal and/or human tumour.

Another aspect of the invention is compounds of general formula I: 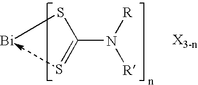
wherein R and R¹ are the same or different, and each is an alkyl, substituted alkyl, aryl or a substituted aryl; X is a halide or a pseudo-halide; and n is either 2 or 3 with the proviso that when n=3, R and R¹ are both not ethyl (Et) and that when n is 3, R is not methyl (Me) and R¹ is not hexyl (Hex) or vice versa.

A further embodiment of the above aspect is novel bismuth dithiocarbamate compounds of general formula II: 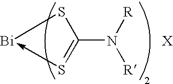
wherein R and R¹ are the same or different, and each is an alkyl, substituted alkyl, aryl or a substituted aryl and X is a halide or a pseudo-halide. The definitions of the terms alkyl, aryl, halide and pseudo-halide are as previously defined. In particular, a novel compound of general formula II, wherein R and R¹ are both (CH₂)₂ and X is Cl.

The process for the preparation of this novel compound is described herein. It involves mixing a bismuth salt, alcohol and carbodithioic acid to obtain a mixture and then dissolving the precipitate obtained from the mixture in a halogen-substituted alkane and organic nitrile to further obtain a solution, followed by drying the said solution to recover an amorphous form of compound of formula II. The reactions are all carried out under a nitrogen atmosphere and at room temperature. All reagent grade solvents are used without further purification.

In particular, the carbodithioic acid is dissolved in distilled water and added to a suspension containing the bismuth salt and alcohol. The resulting mixture is stirred to ensure that reaction between the reagents is complete. The precipitate that is obtained is then separated from the rest of the mixture and washed with distilled water several times. The precipitate that is collected is then dissolved in a halogen-substituted alkane. The solvent is then evaporated and the crude precipitate that remains is recrystallised by using an organic nitrile, with further addition of the halogen-substituted alkane. The end product is crystals of the desired compound. A more detailed description of the method for the production of a particular novel compound, Bi[S₂CN(CH₂)₄]Cl, is provided in Example 1.

Having now generally described the invention, the same will be more readily understood through reference to the following examples which are provided by way of illustration, and are not intended to be limiting of the present invention.

### EXAMPLES

### Example 1

### Preparation of an Example of Bi(S₂CNRR¹)ₙX₃₋ₙ, n=2, R═R¹═(CH₂)₂ and X═Cl

The reactions were all carried out under a nitrogen atmosphere and at room temperature. All reagent grade solvents were used without further purification. The starting materials were obtained commercially such as anhydrous bismuth (III) chloride, BiCl₃ (Strem Chemicals Co), sodium diethyldithiocarbamate trihydrate, (CH₃CH₂)₂NCS₂Na.3H₂O, bismuth nitrate pentahydrate, Bi(NO₃)₃.5H₂O and 1-pyrrolidinecarbodithioic acid ammonium salt, NH₄[S₂CN(CH₂)₄] (Aldrich Chemical Co). ¹H and {¹H} ¹³C NMR spectra were recorded on a Bruker ACF 300 MHz FT NMR spectrometer. Infrared spectra were recorded as KBr discs on an Excalibur Series Bio-Rad Merlin FTS 3000 spectrophotometer in the range of 400-4000 cm⁻¹. Mass spectra were recorded on FINNIGAN TSQ 7000 spectrometer. Elemental analysis was carried out on a Perkin-Elmer PE 2400CHN and CHNS Elemental Analyzer. Melting points were determined on a BUCHI B-540 p apparatus.

NH₄[S₂CN(CH₂)₄] (20.00 mmol, 2.3379 g) dissolved in distilled water (75 ml) was added slowly to a suspension of BiCl₃ (6.78 mmol, 2.1376 g) in ethanol (50 ml). The resultant mixture was stirred for two hours to ensure complete reaction. The bright yellow precipitate was collected using Buchner funnel and washed with distilled water (3×20 ml). The product was then dissolved in chloroform, CHCl₃ (100 ml) and dried-over magnesium sulphate, (MgSO₄). The solvent was later evaporated using a rotary evaporator and the crude yellow precipitate recrystallised using an acetonitrile, C₂H₃N and CHCl₃ mixture (1:1 ratio). Bright yellow crystals of the compound were obtained and characterized as a mono-chloroform solvate. Elemental analysis (%) found: C, 22.16; H, 2.92. Calcd: C, 22.37; H, 3.00. ¹H NMR: δ 3.90 (4 H, t, ³J=6.83 Hz, NCH₂CH₂); δ 2.03 (4 H, m, NCH₂CH₂). {¹H} ¹³C NMR: δ 196.9 (S₂C); 53.3 (NCH₂CH₂); 25.2 (NCH₂CH₂). IR (cm⁻¹): 1479 [ν(C—N)], 991 [ν(C—S)]. MS: m/z=501 (Bi[S₂CN(CH₂)₄]₂⁺). Yield is 62%. Melting point of 230-232° C. (Koh et. al., 2003, Chem. Mater., 15,4544-4554).

### Example 2

The compound, Bi(S₂CNEt₂)₃, is a known and well-characterised species and the crystal structure is known (J. A. Howard et. al., 1975, Acta Crystallogr. A31, S141 and C. L. Raston & A. H. White, 1976, J. Chem. Soc., Dalton Trans, 791) and can be prepared readily and in high purity in the following fashion. All reactions were carried out under a nitrogen atmosphere and at room temperature. All reagent grade solvents were used without further purification. The starting materials, anhydrous bismuth(III) chloride (Strem Chemicals Co) and sodium diethyldithiocarbamate trihydrate, (CH₃CH₂)₂NCS₂Na.3H₂O (Aldrich Chemical Co), were obtained commercially and were used without further purification. ¹H and {¹H}¹³C NMR spectra were recorded on a Bruker ACF 300 MHz FT NMR spectrometer. Infrared spectra were recorded as KBr discs on an Excalibur Series Bio-Rad Merlin FTS 3000 spectrophotometer in the range of 400-4000 cm⁻¹. Mass spectra were recorded on FINNIGAN TSQ 7000 spectrometer. Elemental analysis was carried out on a Perkin-Elmer PE 2400 CHN and CHNS Elemental Analyzer. The melting point was determined on a BÜCHI B-540 mp apparatus.

Other derivatives of the general formula were prepared in the same way and their spectroscopic characteristics determined similarly. The spectroscopic results were entirely consistent with their formulations as Bi(S₂CNRR¹)₃. Crystal structure determinations have also been performed for several of the derivatives, providing further evidence of their chemical composition. A representation of the crystallographically determined structure of Bi(S₂CNEt₂)₃ is shown in J. A. Howard et. al., 1975, Acta Crystallogr. A31, S141 and C. L. Raston & A. H. White, 1976, J. Chem. Soc., Dalton Trans, 791.

A summary of in vitro cytotoxicity for the two representative compounds, namely Bi(S₂CNEt₂)₃ and Bi(S₂CN(CH₂)₄)₂Cl is given in Table 1. The two compounds demonstrated cytotoxicity against the panel of human cancer cell lines comparable to taxol.

TABLE 1
ID₅₀ values (ng/ml) of Bi(S₂CNEt₂)₃, Bi(S₂CN(CH₂)₄)₂Cl, plus
those for standard compounds, doxorubicin (DOX), cisplatin (CPT),
5-fluorouracil (5-FU), methotrexate (MTX), etoposide (ETO) and
taxol (TAX) in vitro using SRB as cell viability test

| Compound | A498 | EVSA-T | H226 | IGROV | M19 | MCF-7 | WIDR |
|---|---|---|---|---|---|---|---|
| Bi(S₂CNEt₂)₃ | 145 | 6 | 10 | <3.2 | 110 | 4 | 56 |
| Bi(S₂CN(CH₂)₄)₂Cl | 70 | 10 | 16 | 5 | 96 | 5 | 23 |
| DOX | 90 | 8 | 199 | 60 | 16 | 10 | 11 |
| CPT | 2253 | 422 | 3269 | 169 | 558 | 699 | 967 |
| 5-FU | 143 | 475 | 340 | 297 | 442 | 750 | 225 |
| MTX | 37 | 5 | 2287 | 7 | 23 | 18 | <3.2 |
| ETO | 1314 | 317 | 3934 | 580 | 505 | 2594 | 150 |
| TAX | <3.2 | <3.2 | <3.2 | <3.2 | <3.2 | <3.2 | <3.2 |

The codes for the cell lines are as follows: A498: renal cancer; EVSA-T: breast cancer, estrogen receptor (ER)−/progesterone receptor (PgR)—; H226: non-small cell lung cancer; IGROV: ovarian cancer; M19 MEL: Melanoma; MCF-7: breast cancer, estrogen receptor (ER)+/progesterone receptor (PgR)+; WIDR: colon cancer.

The results shown in Table 1 were obtained in the following manner. The test and reference compounds were dissolved to a concentration of 250 000 ng/ml in full medium, by 20 fold dilution of a stock solution which contained 1 mg compound/200 μl. The trial complexes were taken into DMSO. Cytotoxicity was estimated by the microculture sulforhodamine B (SRB) test (Keepers, Pizao, et. al., 1991, Eur. J. Cancer, 27, 897).

The experiment was started on day 0. On day 0, 150 μl of trypsinized tumour cells (1500-2000 cells/well) were plated in 96-well flatbottom microtiter plates (falcon 3072, DB). The plates were pre-incubated 48 hrs at 37° C., 8.5% CO₂ to allow the cells to adhere. On day 2, a three-fold dilution sequence of ten steps was made in full medium, starting with the 250 000 ng/ml stock solution. Every dilution was used in quadruplicate by adding 50 μl to a column of four wells. This resulted in a highest concentration of 62 5000 ng/ml being reached in column 12. Column 2 was used as a blank. PBS was added to column 1 to diminish interfering evaporation. On day 7, the incubation was terminated by washing the plate twice with PBS. Subsequently, the cells were fixed with 10% trichloroacetic acid in PBS and placed at 4° C. for one hour. After five washings with tap water, the cells were stained for at least 15 minutes with 0.4% SRB dissolved in 1% acetic acid. After staining, the cells were washed with 1% acetic acid to remove the unbound stain. The plates were air-dried and the bound stain was dissolved in 150 μl 10 mM Tris-base. The absorbance was read at 540 nm using an automated microplate reader (Labsystems Multiskan MS). The data obtained was used for the construction of concentration-response curves and the determination of the ID₅₀ value using the Deltasoft 3 software.

### Example 3

The in vitro cytotoxicity for the two representative compounds, Bi(S₂CNEt₂)₃ and Bi(S₂CN(CH₂)₄)₂Cl was investigated against other cell lines, namely OVCAR, A-498, HF, NCl-H1299, HT-29, MRC-5 and MCF-7, following the same procedure as described in Example 2 above.

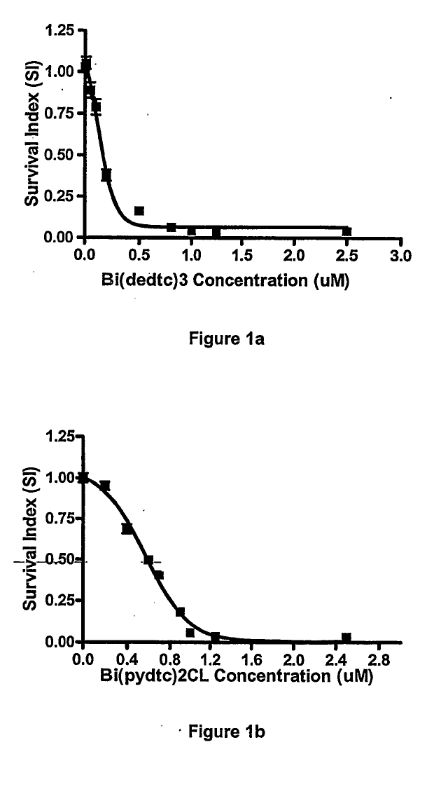

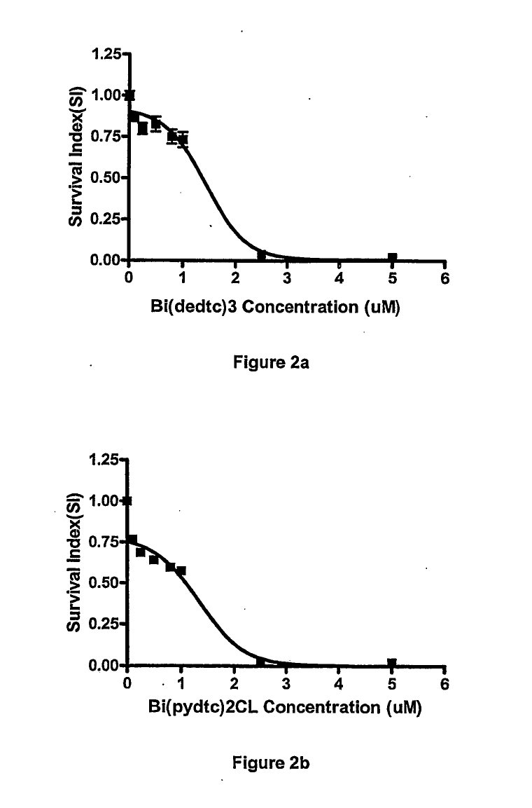

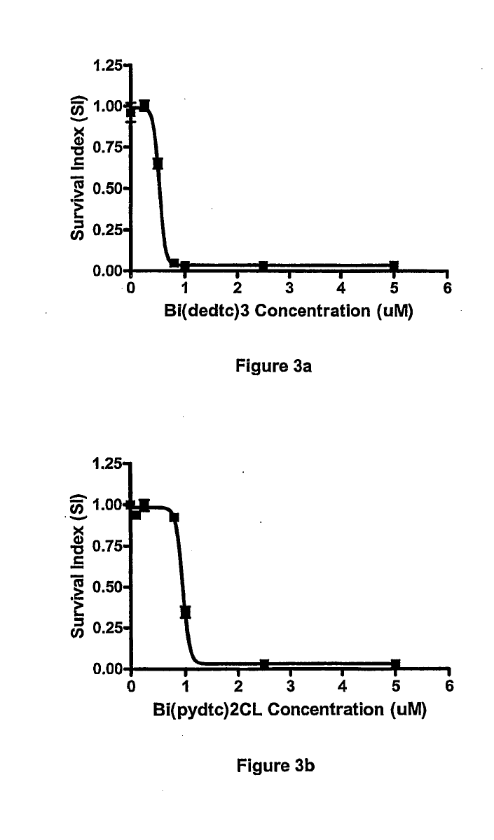

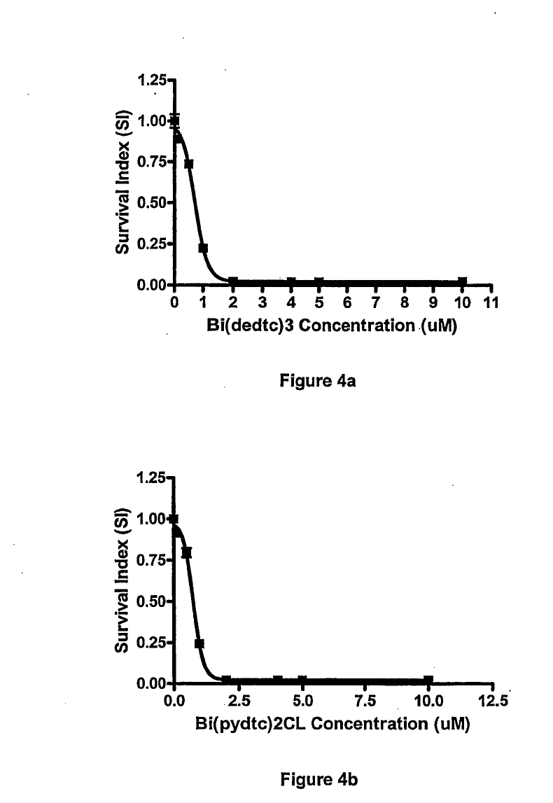

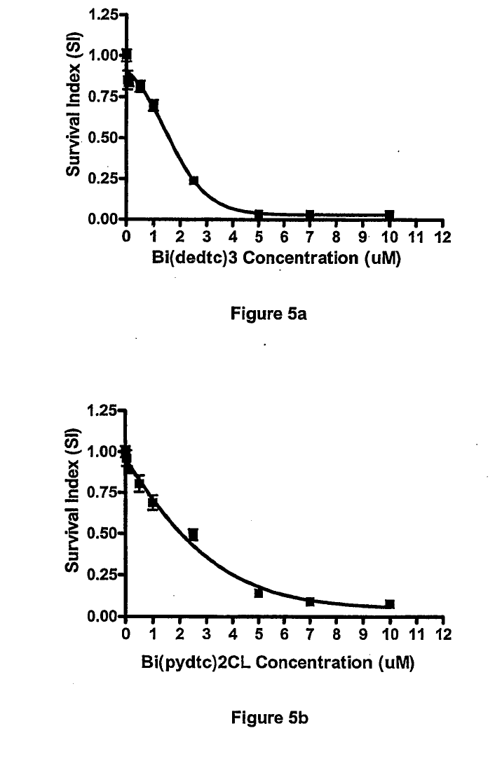

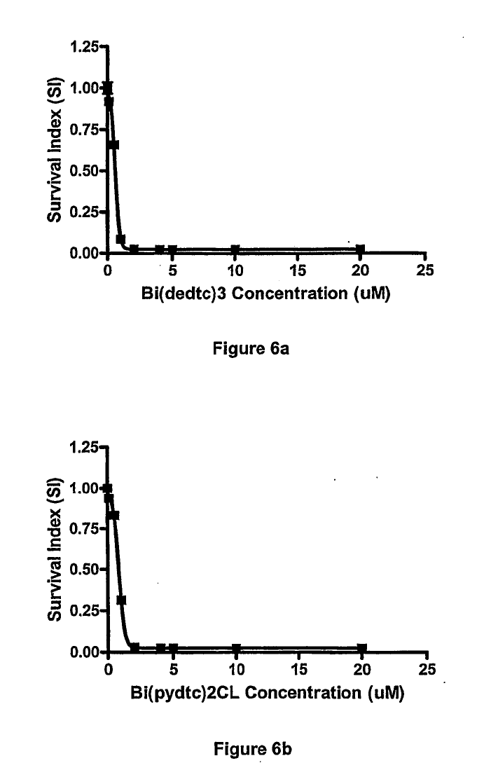

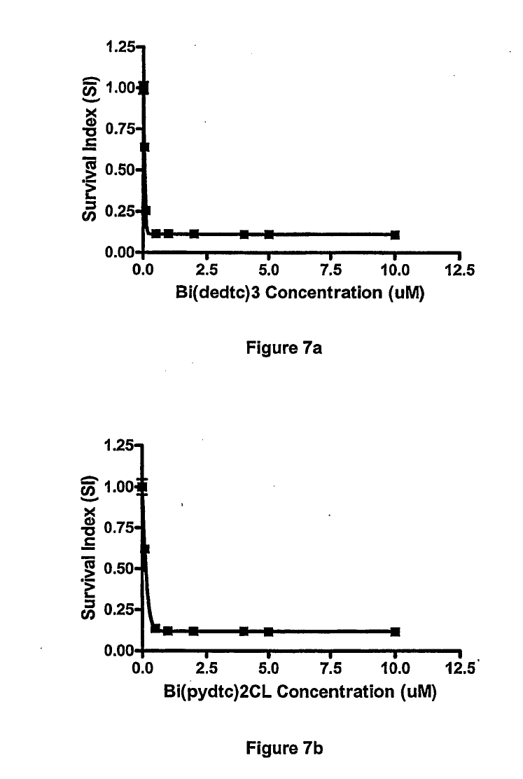

The data obtained was used for the construction of concentration-response curves for the determination of ID₅₀ values using the Deltasoft 3 software. These curves are shown in FIGS. 1-7.

Cell survival is presented as a survival index (SI), which is defined as the absorbance in the experimental wells expressed as a percentage of that in the control wells. The IC50 value is defined as the concentration giving a SI of 50% of the control SI.

### Example 4

The reported in vitro potency is maintained in nude Balb/C mice inoculated with a human ovarian cancer cell line, OVCAR. In order to determine the appropriate dose of Bi(S₂CNEt₂)₃, the Maximum Tolerated Dose (MTD) in Balb/C mouse model was determined.

The MTD of Bi(S₂CNEt₂)₃ was determined in the following manner. Male and female BALB/c mice obtained from the Laboratory Animal Centre, (National University of Singapore), were used for the study. The mice used were about 4-5 weeks old with a mean body weight of 20±2 g. The 5 mice per cage were kept at room temperature and under standard light conditions. They received standard mouse chow and water. The compound Bi(S₂CNEt₂)₃ was dissolved in sterile DMSO and 0.1 ml was given intraperitoneal (i.p.) for a 20 g mouse. The mice in the control group were administered with DMSO only. The concentrations of Bi(S₂CNEt₂)₃ were calculated in terms of mg Bi/kg of mouse. An initial concentration of 20 mg Bi/kg was used for a small sampling population of two mice, one of each gender. If both mice died, the complex concentration was decreased, and the experiment was repeated for another two mice. If one of the mice died but the other survived, the experiment was repeated for a larger sampling population of ten mice, five of each gender, for the same complex concentration. The observation period lasted 14 days. The objective of the experiment was to plot a graph of percent of live mice (number of survivors/ten×100%) against compound dosage (in mg Bi/kg). This procedure enabled the determination of the Maximum Tolerated Dose (at MTD 90% survival of mice).

Using this protocol, the following results were obtained for Bi(S₂CNEt₂)₃:
LD₅₀=3.57 mg/kg
LD₉₀=7 mg/kg
LD₁₀=0.14mg/kg

### Example 5

The anti-tumour activity of Bi(S₂CNEt₂)₃ was determined by in vivo studies. Female BALB/C-nude mice, obtained from the Animal Resources Centre in Queensland, Australia were used for this experiment. The mice used were about 5-6 weeks old and had a mean body weight of 20±2 g. Throughout this experiment, the mice were housed in filtered air laminar-flow cabinets and were manipulated following standard aseptic procedures.

Growth of OVCAR Cancer Cells

Human ovarian cancer (OVCAR) cells were provided by Dr Ho (Department of Pharmacy, National University of Singapore). OVCAR cells were grown in RPMI-1640 medium with 10% foetal bovine serum and 5 mM L-glutamine.

Inoculation of Mice

A suspension of OVCAR cells (10⁷ cells) were injected subcutaneously (s.c.) in the flank of each animal (0.1 ml per mice). When the volume of the tumour reached approximately 50-100 mm³, the mice were randomly divided into 2 groups, the test group and the control group, comprising 6 mice in each group.

Drug Treatment

Bi(S₂CNEt₂)₃ was prepared as a 20 mg Bi/kg stock solution in dimethylsulphoxide, DMSO, and further diluted with DMSO immediately before administration. The compound was given subcutaneously to mice in amounts of 0.1 ml/20 g three times every two days in the first week. The dose applied was at the level of the MTD, i.e. 0.14 mg Bi/kg mouse. The appropriate vehicle (DMSO) was injected into the mice in the control group, using the same schedule and route of injection as the drug therapies.

Treatment Evaluation

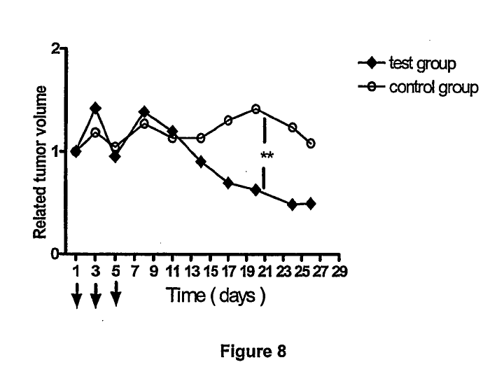

In xenografts transplanted subcutaneously, tumour growth was monitored and the volume of the tumour was determined by measuring its diameter with a vernier calliper every 2-4 days. The Relative Tumour Volume (RTV=tumour volume on day×/tumour volume on day 1) was calculated using the formula width²×length×0.52. The results obtained are shown in FIG. 8.

In summary, the above experiment shows that three weeks after multiple administration of Bi(S₂CNEt₂)₃ to nude Balb/C mice afflicted with OVCAR human ovarian cancer at 0.14 mg Bi/kg animal weight, the average volume of a tumour in a mouse in the test group was 30% that of a mouse in the control group. This shows that Bi(S₂CNEt₂)₃ demonstrates significant anti-tumour activity.

What is claimed is:

1-20. (canceled)

21. A pharmaceutical composition comprising the compound of general formula I: 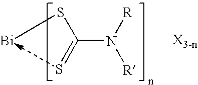
wherein R and R¹ are the same or different, and each is an alkyl, substituted alkyl, aryl or a substituted aryl; X is a halide or a pseudo-halide; and n is either 2 or 3; for use as an active pharmaceutical substance.

22. The pharmaceutical composition of claim 21, further comprising a pharmaceutical acceptable diluent and/or carrier.

23. The pharmaceutical composition of claim 21, wherein n is 3 and R and R¹ are both ethyl.

24. The pharmaceutical composition of claim 22, wherein n is 3 and R and R¹ are both ethyl.

25. A bismuth dithiocarbamate compound of general formula II: 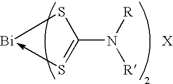
wherein NRR′=N(CH₂)₄, X is Cl.

26. A process for preparing the compound of claim 25, which comprises mixing a bismuth salt, alcohol and carbodithioic acid to obtain a mixture and then dissolving precipitate obtained from the mixture in a halogen-substituted alkane and organic nitrile to further obtain a solution, followed by drying said solution to recover an amorphous form of compound of formula II.

27. The process of claim 26, wherein the bismuth salt is anhydrous bismuth (III) chloride.

28. The process of claim 26, wherein the alcohol is ethanol.

29. The process of claim 26, wherein the carbodithioic acid is 1-pyrrolidinecarbodithioic acid, NH₄S₂CNC₄H₈.

30. The process of claim 26, wherein the halogen-substituted alkane is chloroform, CHCl₃.

31. The process of claim 26, wherein organic nitrile is acetonitrile, C₂H₃N.

32. The process of claim 26, wherein the halogen-substituted alkane and organic nitrile is mixed in a 1:1 volume ratio.

33. A method for treating tumor comprising the administration of compound of general formula I: 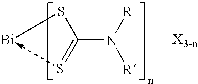
wherein R and R¹ are the same or different, and each is an alkyl, substituted alkyl, aryl or a substituted aryl; X is a halide or a pseudo-halide; and n is either 2 or 3.

34. The method of claim 33, wherein n is 3, R and R¹ are both ethyl.

35. The method of claim 33, wherein the tumor includes breast cancer, ovarian cancer, melanoma, renal cancer and non-small cell lung cancer.

36. The method of claim 33, wherein the tumor is animal and/or human tumor.

37. The method of claim 34, wherein the tumor includes breast cancer, ovarian cancer, melanoma, renal cancer and non-small cell lung cancer.

38. The method of claim 34, wherein the tumor is animal and/or human tumor.
